(19) United States
(12) Patent Application Publication
(10) Pub. No.: US 2012/0231443 A1
(43) Pub. Date: Sep. 13, 2012

(54) MICROCAPSULES FOR CELL MICROENCAPSULATION

(75) Inventors: Xiaoming He, Dublin, OH (US); Wujie Zhang, Columbus, OH (US)

(73) Assignee: University of South Carolina, Columbia, SC (US)

(21) Appl. No.: 13/416,563

(22) Filed: Mar. 9, 2012

Related U.S. Application Data
(60) Provisional application No. 61/464,827, filed on Mar. 9, 2011.

(51) Int. Cl. C12N 11/10

(52) U.S. Cl. 435/2; 435/178

(57) ABSTRACT

In accordance with certain embodiments of the present disclosure, a method for formation of a microcapsule is described. The method includes encapsulating a cell in a microcapsule, the microcapsule having a diameter of less than about 100 μm. The method further includes coating the microcapsule with chitosan and alginate.

### CROSS-REFERENCE TO RELATED APPLICATION

The present application is based on and claims priority to U.S. Provisional Application 61/464,827 having a filing date of Mar. 9, 2011, which is incorporated by reference herein.

### BACKGROUND

Transplantation of encapsulated cells has been proposed to be a promising method for the treatment of a wide variety of diseases such as diabetes, metabolic deficiencies, liver failure, and Parkinson's disease. Moreover, microencapsulated recombinant cells have been used for gene therapy of bone defects and neurodegenerative and cardiovascular diseases.

Small microcapsules offer many advantages over larger ones for transplantation. Reduction in microcapsule size has been reported to promote the molecular exchange between the encapsulated cells and their surrounding environment. In particular, size reduction reduces the resistance to the transport of oxygen and nutrients to the encapsulated cells and enhances transfer of therapeutic products secreted by the cells out of the capsule. Moreover, small microcapsules have been shown to have better mechanical stability and biocompatibility. Remarkably, it has been reported that reduction in microcapsule size suppresses foreign body response to implanted microcapsules.

Alginate is the most widely used biomaterial for cell microencapsulation due to its natural origin (e.g. brown seaweeds) and excellent biocompatibility. Alginate is a linear block polymers made up of α-L-guluronic acid (G) and β-D-manuronic acid (M). The gelation of alginate in divalent cations and formation of Ca alginate microspheres is caused by the formation of the calcium junctions of GG-GG, MG-GG and MG-MG between alginate molecules. Alginate microcapsules can be coated with a polycation layer to increase stability and allow for further control of membrane permeability of the microcapsules. Poly-L-lysine (PLL) is the most studied and employed polycation to produce alginate-PLL-alginate (APA) microcapsules. However, PLL is an inflammatory molecule and likely to be responsible for fibrotic overgrowth, because soluble PLL induces cytokine production in monocytes and can cause cellular necrosis. Chitosan, a biodegradable polysaccharide with structural characteristics similar to glycosaminoglycans, has attracted much attention as a replacement for PLL.

Chitosan is less immunogenic than PLL and has excellent cell affinity. Chitosan, including lactose modified chitosan and oligochitosan (<10 kD), has been applied for microencapsulation of various mammalian cells. However, processes involved with high molecular weight chitosan (>100 kDa) must be carried out below a pH of 6.0 to ensure chitosan solubility, while mammalian cells require a pH between 6.8 and 7.4 for survival. Moreover, the chitosan coating process, using high molar mass chitosan, requires a relatively long coating time of up to 30 min. This prolonged exposure of mammalian cells to low pH results in low cell viability after coating. In addition, the swelling and breaking of alginate-chitosan-alginate (ACA) microcapsules remains an issue when liquefying the core of relatively big alginate-chitosan microcapsules, even when oligochitosans are used. Such difficulties may explain why no study has been reported to successfully synthesize ACA microcapsules for cell microencapsulation.

Therefore, a need exists for a method of synthesis for ACA microcapsules.

### SUMMARY

In accordance with certain embodiments of the present disclosure, a method for formation of a microcapsule is described. The method includes encapsulating a cell in a microcapsule, the microcapsule having a diameter of less than about 100 μm. The method further includes coating the microcapsule with chitosan and alginate.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures in which:

FIG. 1 illustrates a coating process of cell-loaded microspheres in accordance with certain aspects of the present disclosure.

FIG. 2 illustrates images of encapsulated cells with corresponding fluorescence images of the cells after Calcein-AM/EthD-1 staining in accordance with certain aspects of the present disclosure (green for live cells and red for dead cells) (scale bar: 100 μm).

FIG. 3 illustrates the effect of chitosan concentration (a) and incubation time of mannitol solution (b) on cell viability in accordance with certain aspects of the present disclosure (error bar represents standard deviation).

FIG. 4 illustrates images of the microcapsules after chitosan coating under different conditions: without mannitol wash before coating (A), with mannitol wash before chitosan coating (B), 0.1% (C) and 0.5% (D) chitosan solution was used during coating, all in accordance with certain aspects of the present disclosure (scale bar: 100 μm).

FIG. 5 illustrates the effect of chitosan solution on microcapsule size after coating in accordance with certain aspects of the present disclosure (the error bar represents standard deviation).

FIG. 6 illustrates Zeta potential (a) and ATR-FTIR spectra (b) of microcapsules (spheres) in accordance with certain aspects of the present disclosure.

FIG. 7 illustrates typical images of microcapsules (spheres) with corresponding fluorescence images after incubation with FITC labeled probes: Ca alginate microspheres incubated with FITC-IgG (a and b), and ACA microcapsules incubated with FITC-IgG (c and d) and FITC-dextran (e and f) (scale bar: 100 μm) in accordance with certain aspects of the present disclosure.

FIG. 8 illustrates transition of metabolic activities of cells encapsulated in ACA microcapsules (a) typical images of cell-loaded ACA microcapsules (b, d and f) and fluorescence staining of the cells (green for live cells and red for dead cells) (c, e and g) in accordance with certain aspects of the present disclosure (scale bar: 100 μm).

### DETAILED DESCRIPTION

Reference will now be made in detail to embodiments of the disclosed subject matter, one or more examples of which are set forth below. Each example is provided by way of explanation of the subject matter, not limitation of the subject matter. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

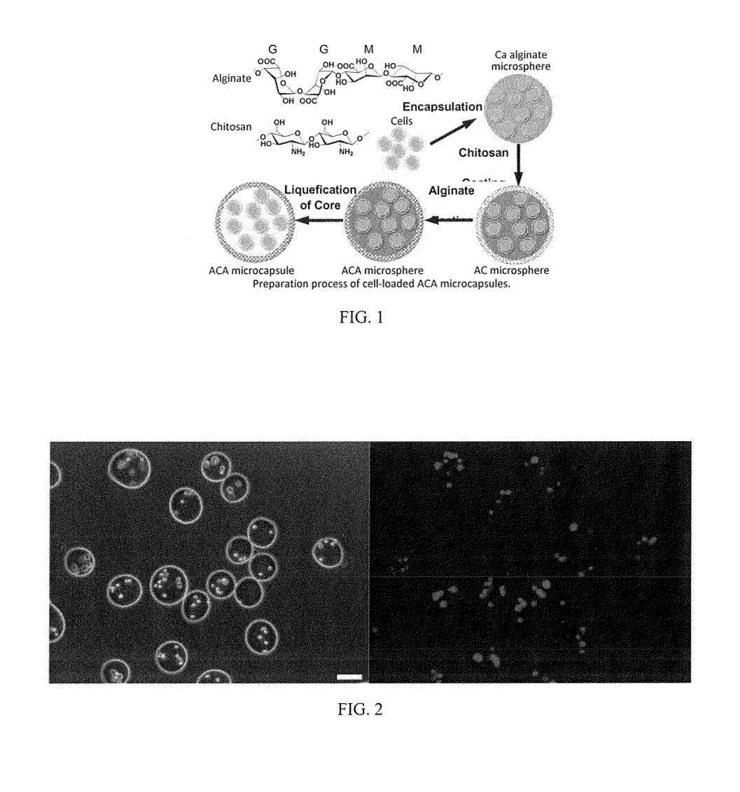

Previously, cells have been successfully encapsulated by the present inventor in small (˜100 μm) Ca alginate microspheres with high cell viability using an electrostatic spray method, as described in U.S. application Ser. No. 12/796,994, incorporate by reference herein. In accordance with the present disclosure, the small microspheres are further coated with low molecular weight chitosan (80 kD) and then alginate to produce ACA microspheres. The core of the microspheres is further liquefied to obtain ACA microcapsules. The coating process of cell-loaded microspheres is shown in FIG. 1.

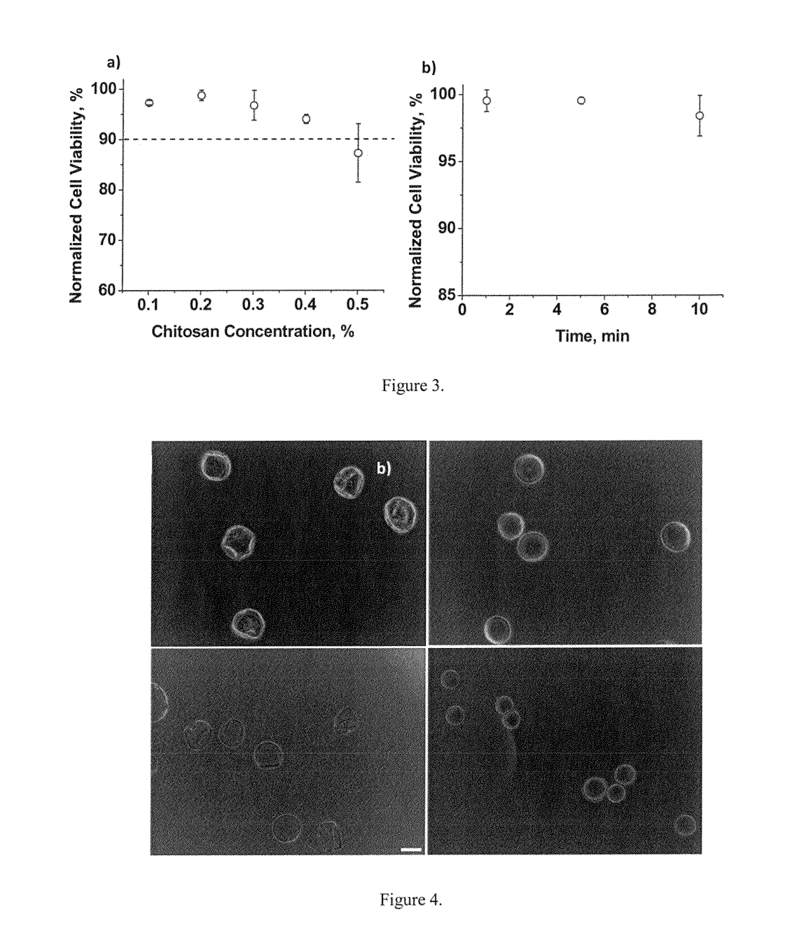

The present disclosure further describes influences on cell viability by the major solutions used for microencapsulation and coating, such as alginate/mannitol and mannitol and chitosan solutions. Alginate (2%, w/v) dissolved in 0.25 M mannitol solution can be utilized for cell microencapsulation. In this manner, cell viability is greatly improved without influencing the microcapsule morphology as compared with previous results. The cell viability in accordance with the present disclosure is 98.5±1.7% after microencapsulation. Typical images of the encapsulated cells with corresponding fluorescence images indicating cell viability are shown in FIG. 2. The toxicity to cells of low molecular weight chitosan, up to 0.5% (w/v), was minimal as seen in FIG. 3A. Moreover, 0.5 M mannitol solution, used for washing microspheres during the ACA microcapsules preparation process, showed no toxicity to cells either as can been seen in FIG. 3B.

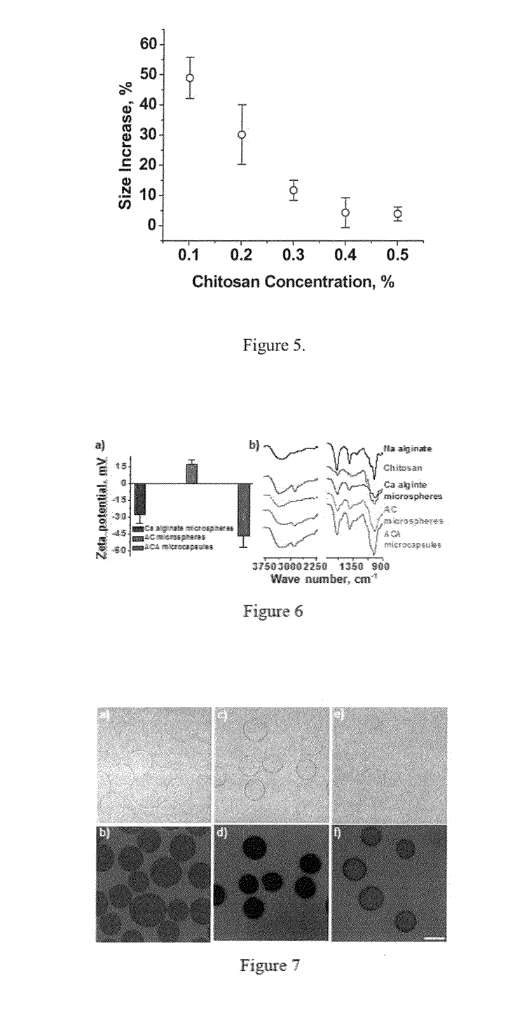

Coating the small alginate microsphere poses difficulties because of microsphere collapse during coating as shown in FIG. 4A. In accordance with the present disclosure, it has been determined that the collapse can be resolved by washing Ca alginate microspheres with mannitol instead of 0.9% (w/v) NaCl solution. As shown in FIG. 4B, ACA microcapsules of round shape with colorful boundary were observed using phase contrast microscopy. Moreover, the mannitol washing time, from 3 to 10 min, before coating did not influence the coating process. The concentration of chitosan solution showed a great influence on the coating process. When low concentration solutions (<0.3%, w/v) were used, the microcapsules became swollen as seen in FIG. 5, and were easily broken after liquefying the Ca alginate core as shown in FIG. 4C. However, the microcapsules tended to be stuck to one another when the concentration of chitosan solution was higher than 0.4% as shown in FIG. 4D. Moreover, the chitosan coating time, from 3 to 10 min, showed no significant effects on microcapsule size and morphology. The optimized coating conditions for high cell viability and good microcapsule morphology was found to be the following: mannitol wash (3-10 min) before coating, 0.4% (w/v) chitosan solution, and 3-min-long coating time.

ACA microcapsules prepared under the optimal conditions were characterized using both Zeta potential analyzer and ATR-FTIR, and the results are shown in FIGS. 6A and B, respectively. The alginate-chitosan (AC) microspheres give a positive Zeta potential, while it is negative for Ca alginate microspheres, indicating the successful coating of chitosan (polycation) over the microspheres. The ACA microcapsules show a negative Zeta potential, presumably due to successful coating of alginate over the chitosan. The successful preparation of ACA microcapsules was further confirmed by the ATR-FTIR data as shown in FIG. 1B). The spectra of the AC microspheres and ACA microcapsules display characteristic peaks of both alginate and chitosan as shown in TABLE I below.

TABLE I
ART-FTIR bands with assignments

| Vibration | | |
|---|---|---|
| (cm⁻¹) | Na alginate | Chitosan |
| 3700-3000 | OH stretch | |
| 3360 | | O—H and N—H stretch |
| 3000-2850 | CH stretch | |
| 2873 | | C—H stretch |
| 1657 | | amide I |
| 1600 | Antisymmetric COO⁻ stretch | |
| 1590 | | N—H bending from amine |
| | | and amide II |
| 1420 | | —CH₂ bending |
| 1410 | symmetric COO⁻ stretch | |
| 1376 | | CH3 symmetrical deformation |

The peaks in the 1200-950 cm-1 region that correspond to various vibrations of the carbohydrate ring, the building blocks of both alginate and chitosan, could be clearly observed. The antisymmetric carbonyl vibration at 1600 cm-1 of the sodium alginate has been reported to be sensitive to the presence of cross linking agents, such as Ca2+ and Ba2+. This band shifted to 1592 cm-1 (right shift) for the Ca alginate microsphere, which was caused by an increase of the ionic interaction strength (replacement of Na+ by Ca2+). On the contrary, this band shifted significantly from 1587 (AC microsphere) to 1595 (ACA microcapsule) cm-1 (left shift), presumably was due to the exchange of Ca2+ with Na+ and alginate coating. Moreover, a new band near 1538 cm-1 of the AC microsphere and ACA microcapsule spectra appears that may be due to the formation of alginate-chitosan complex after chitosan coating.

The permeability of the microcapsules(spheres) was tested with two probes, FITC-IgG (160 kD) and FITC-dextran (4.4 kD). FITC-IgG was used to test the immunoisolation capability (blocking IgG) of the microcapsules, while the FITC-dextran was employed to verify the transport of small molecules (e.g. glucose, amino acids and other small molecules which are important for cell survival and proliferation). For the Ca alginate microspheres, the green fluorescence can be seen (diffusion of the FITC-IgG into the microspheres) within the microspheres (FIGS. 7A and B) even just after 1 h incubation with FITC-IgG. In addition, the size of the Ca alginate microcapsules increased almost 50% in 0.9% (w/v) NaCl solution. However, the ACA microcapsule is impermeable to IgG even after 24 h incubation (FIGS. 7C and D). Moreover, the ACA microcapsule does allow free diffussion of the small FITC-dextran in just 1 h incubation (FIGS. 7E and F). These results indicate that ACA microcapsules could be used to achieve immunoisolation while allowing free diffussion of nutrients and metabolites/therapeutic products.

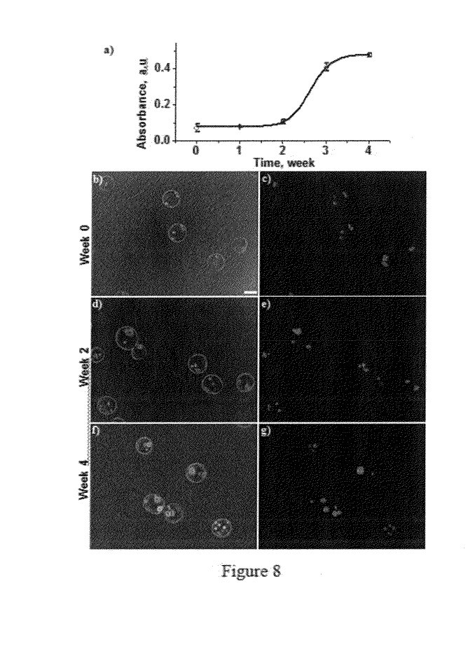

Cell-loaded ACA microcapsules were further prepared under the optimized conditions. High cell viability, 97.9±2.2%, was achieved and the microcapsules were round in shape demonstrating that the presence of cells did not affect the coating process. To test the long-term survival of the encapsulated cells, WST-1 assay was used to monitor the metabolic activities of the cells, as shown in FIG. 8A. The metabolic activity increased (results of cell proliferation), by 6 fold, during the 1 month culture time. Typical images of the encapsulated cells at various time are shown in FIGS. 8B, D and F, and the cell viability was high (dead cells could be barely observed, FIGS. 8C, E and G) during the whole test period. Cell proliferation was also be confirmed by the formation of visible cell clusters (started in the second week). Both the size and number of the cell clusters increased in the latter weeks. In addition, the microcapsules maintained their round shape without swelling. Together, these data indicate that the encapsulated cells can survive and proliferate well within the small ACA microcapsules.

As such, chitosan coating of small (˜100 μm) cell-loaded ACA microcapsules can be achieved in accordance with the present disclosure. The membrane of the microcapsules was found to be selectively permeable. More importantly, the encapsulated cells were shown to survive well during a 1-month-long extended culture. Therefore, the small ACA microcapsule is a promising system to encapsulate non-autologous cells for cell-based medicine.

Reference now will be made to exemplary embodiments of the invention set forth below. Each example is provided by way of explanation of the invention, not as a limitation of the invention.

### Example 1

Materials:

Purified sodium alginate (type M) was purchased from Medipol (Lausanne, Switzerland). Chitosan of pharmaceutical grade (MW: 80 KD, deacetylation degree 95%) was obtained from Weikang Biological Products Co. Ltd (Shanghai, China). The Viability/Cytotoxicity kit for mammalian cells and the WST-1 cell proliferation reagent were purchased from Invitrogen (Carlsbad, Calif.) and Roche Diagnostics (Mannheim, Germany), respectively. Fluorescein isothiocyanate-immunoglobulin G (FITC-IgG), FITC-dextran and all other chemicals were purchased from Sigma (St. Louis, Mo.) unless specifically indicated otherwise.

Cell Culture and Microencapsulation:

Mouse mesenchymal stromal (C3H10T1/2) cells (ATCC, Manassas, Va.) derived from mouse embryos were used as the cell model for this study since they have been frequently used as the carrier of various genes for gene therapy. The C3H10T1/2 cells were cultured in DMEM (Invitrogen, Carlsbad, Calif.) with 10% fetal bovine serum, 100 U/mL penicillin and 100 mg/L streptomycin (Hyclone, Logan, Utah) at 37° C. in a humidified 5% CO₂ incubator. The C3H10T1/2 cells were detached from culture dishes with trypsin/EDTA digestion (Invitrogen, Carlsbad, Calif.), centrifuged for 3 minutes at 960 rpm, and resuspended in a 0.9% (w/v) sodium chloride solution for cell microencapsulation studies. Cells were centrifuged and re-suspended in 2% (w/v) sodium alginate solution with 0.25 M mannitol. The cell suspension was then transferred to a 3 mL syringe for generating cell-loaded microcapsules using the electrostatic spray method. All the solutions for cell microencapsulation were buffered using 10 mM HEPES to maintain the pH between 7.2 and 7.4. The same alginate solution but without cells was used to prepare non cell-loaded Ca alginate microspheres using the same method.

Preparation of Alginate-Chitosan-Alginate (ACA) Microcapsules:

Ca alginate microspheres obtained using the method previously mentioned were suspended in chitosan solution (pH 6.5-6.6) for various times to form alginate-chitosan microspheres (AC). The AC microspheres, collected by centrifugation, were then suspended in a 0.15% (w/v) sodium alginate solution to counteract the rudimental charges of the coating chitosan. Lastly, the solid core of the microspheres was liquefied by suspending the microspheres in a 55 mM sodium citrate solution for 5 min to obtain alginate-chitosan-alginate (ACA) microcapsules. As a result, the alginate-chitosan-alginate complex layer formed the microcapsule membrane.

Characterization of Alginate Microcapsules:

Microcapsules (spheres) were suspended in de-ionized (DI) water and their surface charges were measured quickly before samples sinking down using a Brookhaven Zeta potential analyzer (Holtsville, N.Y.). The chemistry of the microcapsules (spheres) was studied using ATR-FTIR (Spectrum 100, PerkinElmer), for which the samples were suspended in DI water and freeze-dried before analysis. For the microcapsule membrane permeability test, the microcapsules were added into 2 ml of 0.05% (w/v) FITC-IgG (IgG MW: 160 KD) or FITC-dextran (MW: 4.4 KD) solution and incubated for either 1 or 24 hr. Afterwards, the microcapsules were studied using a Zesis LSM 510 META confocal laser scanning microscope to examine the distribution of the fluorescence probes.

Cell Viability and Proliferation:

Cell viability was determined using the standard live/dead assay kit. The cells were stained and images were taken using an Olympus BX 51 microscope equipped with fluorescent cubes and a QICAM CCD digital camera (QImaging, Surrey, BC, Canada). Green (live cells) and red (dead cells) fluorescence images were collected separately and merged using NIH ImageJ to cell viability (ratio of live cells to total cells). The normalized cell viability, absolute cell viability to fresh control cells, was used in this study. To investigate the long-term survival and proliferation of encapsulated cells in small ACA microcapsules, encapsulated cells were seeded in cell culture inserts (BD Falcon) and used with their companion plates. The medium was renewed outside the inserts to avoid microcapsule loss every 2-3 days. WST-1 assay was performed to measure the encapsulated cell metabolic activity and the encapsulated cells were stained as described previously and observed under the microscope every week for 1 month.

Small (˜100 μm) Alginate-Poly-I-lysine-Alginate (APA) microcapsules:

Ca alginate microspheres were washed for 10 min using 0.5 M mannitol solution and suspended in 0.75% (w/v) PLL (Mw: 30,000˜70,000 D) solution for 2 min under gently shaking. After rinsing microcapsules with 0.5 M mannitol solution, 0.15% (w/v) sodium alginate solution was added, for 3 minutes, to counteract the rudimental charges on the membrane. Lastly, the beads were then liquefied using 0.055 mol/L sodium citrate for 5 min to obtain alginate-PLL-alginate microcapsules.

Small (˜100 μm) Ba Alginate-Chitosan-Alginate (ACA) microcapsules:

Ba alginate microspheres obtained were washed for 5 min using 0.5 M mannitol and then suspended in 0.4% (m/v) chitosan (pH 6.5-6.6) for 3 tim to form alginate-chitosan microspheres (AC). The AC microspheres, collected by centrifugation, were then suspended in a 0.15% (w/v) sodium alginate solution to counteract the rudimental charges of the coating chitosan. Lastly, the solid core of the microspheres was liquefied by suspending the microspheres in a 55 mM sodium citrate solution for 5 min to obtain alginate-chitosan-alginate (ACA) microcapsules. As a result, the alginate-chitosan-alginate complex layer formed the microcapsule membrane.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed:

1. A method for formation of a microcapsule comprising:
encapsulating a cell in a microcapsule, the microcapsule having a diameter of less than about 100 μm;
coating the microcapsule with chitosan and alginate.

2. The method of claim 1, wherein the cell is a mammalian cell.

3. The method of claim 1, wherein the microcapsule comprises a biocompatible material.

4. The method of claim 1, wherein the microcapsule comprises alginate.

5. The method of claim 1, wherein said cell is an oocyte, a sperm, a stern cell, an embryo, or a zygote.

6. The method of claim 1, further comprising washing the microcapsule.

7. The method of claim 6, wherein the microcapsule is washed in mannitol.

8. The method of claim 1, wherein the alginate is utilized to coat the microcapsule after the chitosan is utilized to coat the microcapsule.

9. The method of claim 1, wherein the microcapsule is selectively permeable.

10. The method of claim 1, wherein the microcapsules are formed by utilizing electrostatic spray methods.

11. The method of claim 1, wherein the microcapsule comprises an alginate chitosan alginate microcapsule.

12. A method for formation of a microcapsule comprising:
encapsulating a cell in a microcapsule comprising alginate, the microcapsule having a diameter of less than about 100 μm;
coating the microcapsule with chitosan and alginate.

13. The method of claim 12, wherein the cell is a mammalian cell.

14. The method of claim 12, wherein the microcapsule comprises a biocompatible material.

15. The method of claim 12, wherein said cell is an oocyte, a sperm, a stem cell, an embryo, or a zygote.

16. The method of claim 12, further comprising washing the microcapsule.

17. The method of claim 16, wherein the microcapsule is washed in mannitol.

18. The method of claim 12, wherein the alginate is utilized to coat the microcapsule after the chitosan is utilized to coat the microcapsule.

19. The method of claim 12, wherein the microcapsule is selectively permeable.

20. The method of claim 12, wherein the microcapsules are formed by utilizing electrostatic spray methods.
